(19) United States
(12) Patent Application Publication
(10) Pub. No.: US 2006/0171962 A1
(43) Pub. Date: Aug. 3, 2006

(54) SARS vaccine

(75) Inventors: Luis Enjuanes Sanchez, Madrid (ES); Fernando Almazan Toral, Madrid (ES)

Correspondence Address: BINGHAM, MCCUTCHEN LLP, THREE EMBARCADERO CENTER, 18 FLOOR, SAN FRANCISCO, CA, 94111-4067, US

(73) Assignee: Consejo Superior de Investigaciones Cientificas, Madrid (ES)

(21) Appl. No.: 11/217,337

(22) Filed: Sep. 2, 2005

(30) Foreign Application Priority Data
Sep. 3, 2004 (EP) 04021065.0

(51) Int. Cl. A61K 39/215; C07K 14/165

(52) U.S. Cl. 424/221.1; 530/350

(57) ABSTRACT

The present invention is directed to vaccines comprising a virus particle comprising a nucleic acid and one or several coat proteins, wherein (a) the nucleic acid encodes SARS-CoV replicase, SARS-CoV N protein and at least one further SARS-CoV protein; and (b) the one or several coat proteins are SARS-CoV coat proteins or proteins with a homology of at least 60% to SARS-CoV coat proteins necessary to allow the virus particle to infect human or animal cells, wherein the nucleic acid is replication competent but not infectious.

The present application claims priority to European Patent Application No. 04021065.0, filed Sep. 3, 2004 and is incorporated herein by reference in its entirety.

The present invention is directed to virus particles, wherein these virus particles comprise a nucleic acid and one or several coat proteins. The nucleic acid within the virus particle encodes SARS-CoV replicase, SARS-CoV N protein and at least one further SARS-CoV protein. The one or several coat proteins of the virus particle are SARS-CoV coat proteins necessary to allow the virus particle to infect human or animal cells. The virus particles are further replication competent but not infectious.

### TECHNICAL BACKRGOUND

Therapy approaches that involve the insertion of a functional gene into a cell to achieve a therapeutic effect are also referred to as gene therapy approaches, as the gene serves as a drug. Gene therapy is a technique primarily for correcting defective genes responsible for disease development.

A carrier molecule also referred to as a vector is used to deliver the therapeutic gene to the patient's target cells. Currently, the most common vector is a virus that has been genetically altered to carry human or animal genes. Viruses have evolved a way of encapsulating and delivering their genes to human or animal cells in a pathogenic manner. Scientists have taken advantage of this capability and manipulate the virus genome to remove disease-causing genes and insert therapeutic genes.

Target cells such as the patient's liver or lung cells are infected with the viral vector. The vector then unloads its genetic material containing the therapeutic gene into the target cell. The generation of a functional protein product from the therapeutic gene restores the target cell to a normal state.

In an alternative approach, these viral vectors were used for expressing heterologous genes that cause an immunogenic response in the subject receiving the vector and thus immunize that subject. In that case the viral vector serves as a vaccine.

Since the etiologic pathogen causing SARS was identified as a new coronavirus (Drosten et al., 2003; Holmes and Enjuanes, 2003; Marra et al., 2003; Rota et al., 2003), the study of coronavirus molecular biology was given a very high priority in order to develop effective strategies to prevent and control coronavirus infections.

Coronaviruses are ssRNA(+) viruses which have the largest genome so far found in RNA viruses with a length between 25 and 31 kilobases (kb; see Siddell S. G. 1995, The Coronaviridae). When a coronavirus infects a cell, the genomic RNA (gRNA) replicates in the cytoplasm and a set of subgenomic RNAs (sgRNA) of positive and negative polarity is produced (Sethna et al., 1989; Sawicki & Sawicki, 1990; and Van der Most and Spaan, The Coronaviridae).

Due to the fact that the coronaviruses replicate in the cytoplasm, use of coronaviruses as a vector for gene therapy and vaccination has been suggested. Specifically, defective interfering (DI) genomes of coronaviruses were produced. These DI genomes are deletion mutants which require the presence of a complementing or helper virus for replication and/or transcription (see Chang et al., 1994; WO97/34008; Spanish patent application P9600620; Izeta et al., 1999; and Sanchez et al., 1999).

A respective system was used in the art to generate immune responses in an animal which received a composition containing a DI genome which amongst others contained sequences encoding a heterologous reporter gene or a gene derived from a different infectious agent (porcine reproductive and respiratory disease virus, PRRSV; see Alonso et al., 2002a, 2002b).

The construction of coronavirus full-length cDNA clones (Almazán et al., 2000; Casais et al., 2001; Thiel et al., 2001; Yount et al., 2000; Yount et al., 2002; Yount et al., 2003) provided the opportunity for the genetic manipulation of coronavirus genomes to study fundamental viral processes and to develop expression vectors.

Although SARS-CoV emerged only in 2002, genome sequences of SARS-CoV isolates have been published recently and provide important information on the organization, phylogeny and variability of the SARS-CoV genome (Marra et al., 2003; Rota et al., 2003). About two-thirds of the 29.7-kb Urbani strain genome encode the replicase gene that comprises open reading frames Rep 1a and Rep 1b, the latter one being expressed by ribosomal frameshifting (Thiel et al., 2003). Translation of both ORFs results in the synthesis of two large polyproteins that are processed by viral proteinases to yield the replicase-transcriptase complex (Ziebuhr et al., 2000).

The 3′ one-third of the genome includes the genes encoding the structural and nonstructural proteins, in the order 5′-S-3a-3b-E-M-6-7a-7b-8a-8b-9b-N-3′. These proteins are expressed by a discontinuous transcription process that most probably takes place during the synthesis of the negative strand, leading to the generation of a 3′ coterminal nested set of subgenomic mRNAs, each of which has at its 5′ end a capped leader sequence derived from the 5′ end of the genome (Sawicki and Sawicki, 1998; Zuniga et al., 2004). Synthesis of subgenomic negative sense RNA species is regulated by the transcription-regulating sequences (TRSs), that include a highly conserved core sequence that is found preceding each gene and at the 3′ end of the leader sequence (Thiel et al., 2003).

SARS-CoV first broke out in humans in late 2002 and spread within a few months from its origin in Guangdong (China) to more than 30 countries. The rapid transmission and high mortality rate made SARS a global threat for which no efficacious therapy is available. The problem underlying the present invention thus resides in providing vaccines for protection against SARS with good safety and immunogenicity.

### SUMMARY OF THE INVENTION

The present invention provides vaccines comprising a virus particle, wherein the virus particle comprises a nucleic acid and one or several coat proteins. The nucleic acids encode SARS-CoV replicase, SARS-CoV N protein and at least one further SARS-CoV protein and are replication competent but not infectious. The coat proteins are SARS-CoV coat proteins necessary to allow the virus particle to infect human or animal cells.

A virus particle comprising all coat proteins of a virus and a replication competent nucleic acid not encoding all proteins necessary for infection is also referred to as a pseudovirus in the art. The vaccine can be administered to a human or an animal to reduce or eliminate the symptoms of a subsequent SARS virus infection.

According to another aspect of the present invention the further SARS-CoV proteins encoded by the nucleic acid are proteins capable of associating into SARS-CoV virus like particles.

The vaccines of the present invention may be administered to a mammal to produce an immune response that reduces or eliminates the disease symptoms caused by infection with the SARS virus. The vaccines of the present invention may further comprise pharmaceutically acceptable carrier and/or adjuvants.

### DETAILED DESCRIPTION OF THE INVENTION

The present invention is directed to a vaccine comprising virus particles comprising a nucleic acid and one or several coat proteins, wherein:
(a) the nucleic acid encodes SARS-CoV replicase, SARS-CoV N protein and at least one further SARS-CoV protein; and
(b) the one or several coat proteins are SARS-CoV coat proteins or proteins with a homology of at least 60% to wild type SARS-CoV coat proteins necessary to allow the virus particle to infect human or animal cells,
   wherein the nucleic acid is replication competent but not infectious.

The present inventors have surprisingly found that a vaccine comprising a virus particle as defined above is safe and can initiate an immune response against SARS viruses. The vaccine comprises a virus particle that contains all elements necessary to infect human or animal cells. Upon infection of a cell, the virus will be able to replicate but will no longer be able to produce an infective virus particle, as the nucleic acid of the virus particle comprises less than all genes necessary to produce an infectious virus particle. Replication of the viral nucleic acid is obtained by the viral replicase, however, replication is significantly increased by the additional presence of the viral N protein. The nucleic acid of the virus particle further encodes at least one SARS protein that will be expressed. The high copy number of the nucleic acid obtained due to the presence of the replicase and N protein will increase expression of the at least one further SARS protein. Expression of the at least one further SARS protein is suitable to generate an immune response in a mammal vaccinated with the vaccines of the present invention.

“Replication competent” nucleic acid as used herein means that the nucleic acid replicates itself autonomously when introduced in a cell. The nucleic acid thus comprises sequences encoding all proteins necessary for replication. SARS-CoV coat proteins are proteins, such as the SARS-CoV E, M or S (spike) protein or proteins. The proteins may have a sequence as observed in a SARS isolate or may be derived therefrom by methods known in the art. Using methods of recombinant expression it is for example possible to modify protein sequences by addition, deletion or substitution of one or several amino acids. The present invention thus covers viruses comprising or encoding coat proteins with a sequence homology of at least 60%, preferably at least 75% and most preferably at least 95% to the wild type SARS-CoV proteins.

For the purposes of the present application sequence homology is determined using the Clustal computer program available from the European Bioinformatics Institute (EBI), unless otherwise stated.

In accordance with the present invention vaccines are provided, which are preferably capable of inducing both a systemic immune response and a mucosal immune response against SARS-CoV.

In one embodiment of the present invention the SARS-CoV replicase is encoded by SEQ ID NO:1 (Rep1a) and SEQ ID NO:2 (Rep1b) or a nucleic acid having a homology of at least 60%, preferably at least 75% and most preferably at least 95% to SEQ ID NOs:1 or 2. SEQ ID NOs:1 and 2 contain a partial overlap at the beginning of SEQ ID NO:2, as the Rep1b open reading frame is translated after a ribosomal framshift of −1. The replicase gene may thus be encoded by a single nucleic acid, wherein the overlapping sequence is present only once or by two seperate sequences as shown in SEQ ID Nos: 1 and 2.

In another embodiment the SARS-CoV N protein is encoded by SEQ ID NO:3 or a nucleic acid having a homology of at least 60%, preferably at least 75% and most preferably at least 95% to SEQ ID NO:3.

In a preferred embodiment of the present invention the nucleic acid further encodes the SARS-CoV spike protein. In a more preferred embodiment the SARS-CoV spike protein is encoded by SEQ ID NO:4 or a nucleic acid having a homology of at least 60%, preferably at least 75% and most preferably at least 95% to SEQ ID NO:4.

In a most preferred embodiment the nucleic acid does not encode any other protein except for SARS-CoV replicase, SARS-CoV N protein and SARS-CoV spike protein.

Similar to other coronaviruses, the spike protein of the virus interacts with a cellular receptor and mediates membrane fusion to allow viral entry into susceptible target cells. Accordingly, the spike protein plays an important role in virus infection cycle and is the primary target of neutralizing antibodies.

In another preferred embodiment of the present invention the nucleic acid further encodes the SARS-CoV M protein. In a more preferred embodiment the SARS-CoV M protein is encoded by SEQ ID NO:5 or a nucleic acid having a homology of at least 60%, preferably at least 75% and most preferably at least 95% to SEQ ID NO:5.

In the most preferred embodiment the nucleic acid does not encode any other protein except for SARS-CoV replicase, SARS-CoV N protein and SARS-CoV M protein.

In yet another preferred embodiment of the present invention the nucleic acid further encodes the SARS-CoV E protein. In a more preferred embodiment the SARS-CoV E protein is encoded by SEQ ID NO:6 or a nucleic acid having a homology of at least 60%, preferably at least 75% and most preferably at least 95% to SEQ ID NO:6.

In a most preferred embodiment the nucleic acid does not encode any other protein except for SARS-CoV replicase, SARS-CoV N protein and SARS-CoV E protein.

The M and E protein are structural proteins which are part of the lipid envelope of coronaviruses.

The invention is also directed to vaccines, wherein the nucleic acid further encodes SARS-CoV M protein and SARS-CoV E protein, or SARS-CoV M protein and SARS-CoV spike protein, or SARS-CoV E protein and SARS-CoV spike protein or SARS-CoV M protein, SARS-CoV E protein and SARS-CoV spike protein. These combinations may also be encoded by nucleic acid sequences having at least 60%, preferably at least 75% and most preferably at least 95% homology to the SARS-CoV wild type nucleic acid sequences.

In another preferred embodiment of the invention the nucleic acid further encodes SARS-CoV proteins capable of associating into SARS-CoV virus like particles.

In the present application the term “virus-like particles” (or VLPs) is used to refer to an association of several proteins of one virus that resembles the virus. The VLPs do not contain any nucleic acid. The proteins may be covalently coupled or otherwise linked and may have the same or a different sequence. Preferably the particle comprises an association of several different viral coat proteins.

While the nucleic acids may encode any SARS-CoV protein capable of associating into a VLP, in a preferred embodiment of the present invention they encode SARS-CoV proteins M, SARS-CoV E protein and SARS-CoV spike protein or proteins with a homology of at least 60%, preferably at least 75% or most preferably at least 95% to these proteins, wherein SARS-CoV M, E and spike proteins form virus-like particles.

These VLPs will initiate immune responses closely mimicking the immune response caused by the wild-type virus particle.

In another embodiment of the invention the nucleic acid further encodes co-stimulating molecules, for example CD80 or CD86 or immunostimulating cytokines, for example TNF-alpha, IFN-gamma, granulocyte/macrophage colony-stimulating factor, interleukin-2 (IL-2), IL-12, IL-18 or CpG.

The vaccine of the present invention may be administered to a mammal to obtain an immune response that reduces or eliminates the disease symptoms caused by infection with the SARS virus. The vaccine is preferably used for vaccinating a mammal, especially a human.

The vaccine may be administered in accordance with methods routinely used in the art. Specifically vaccine may be administered by intramuscular, intravenous, subcutaneous or intranasal administration.

The vaccines may also comprise pharmaceutically acceptable carriers, excipients and/or adjuvants.

Adjuvants and carriers suitable for administering genetic vaccines and immunogens via the mucosal route are known in the art. Conventional carriers and adjuvants are for example reviewed in Kiyono et al. 1996. The addition of chemokines that are used to modulate immune responses are also encompassed by the present invention. Respective compounds and their medical use has been reviewed in Toka et al. 2004. It is specifically advantagous to use one of granulocyte/macrophage colony-stimulating factor, interleukin-2 (IL-2), IL-12, IL-18. Combinatorial approaches utilizing several cytokines and chemokines might also be applied. In addition, as more is discovered regarding the requirements for memory development of T cells, boosters involving key cytokines such as IL-15 and IL-23 may prove beneficial to long-term maintenance of the memory pool.

The present invention is also directed to nucleic acids, wherein these nucleic acids are SARS-CoV replicons. The term “Replicon” is used in the present application to refer to a nucleic acid capable of autonomous replication, the nucleic acid thus encodes all proteins that are needed to replicate the nucleic acid upon inserting the nucleic acid into a host cell. The nucleic acid may further comprise other SARS sequences, but is preferably non-infectious, i.e. does not encode proteins that are capable of associating with the nucleic acid to form a virus particle that is capable of infecting other cells.

In one embodiment the replicon encodes SARS-CoV replicase and SARS-CoV N protein. In a preferred embodiment the replicon encodes at least one further SARS-CoV protein, preferably the SARS-CoV spike protein. The invention also comprises replicons, wherein the nucleic acids encoding SARS-CoV proteins have a homology of at least 60%, preferably at least 75% and most preferably at least 95% to the SARS-CoV wild-type nucleic acids.

Methods to prepare the nucleic acids and the virus particles of the present invention may comprise the preparation of a SARS-CoV derived replicon in a bacterial artificial chromosome (BAC). The SARS sequences may be based on the Urbani strain genome (Genebank, accession number AY278741). The virus particles in the vaccines of the present invention are preferably prepared starting from a SARS replicon.

A replicon can be generated using a set of overlapping cDNAs spanning the untranslated 5′ and 3′ ends of the genome, the entire replicase gene, and the nucleoprotein (N) gene.

The strategy for the construction of the SARS-CoV replicon as a bacterial artificial chromosome (BAC) can be summarized as follows:
(i) Identification of appropriate restriction sites in the viral genome to be used in the engineering of the replicon.
(ii) Generation of an intermediate plasmid as the basis for construction of the SARS-CoV replicon.
(iii) Generation of overlapping SARS-CoV cDNA subclones by RT-PCR and replicon assembly using the restriction sites selected.

The construction of a SARS-CoV replicon cDNA is illustrated with further details in Example 1.

Methods for preparing the virus particles used in the vaccines of the present invention may use a replicon as described above. According to a preferred aspect of the present invention these methods are based on the use of the plasmid pBAC-SARS-CoV-REP. Bacteria containing this plasmid were deposited on Sep. 1, 2004 with the Colección Españnola de Cultivos (Tipo, CECT in Valencia, Spain). The deposit was given the provisional accession number 7020.

The plasmid pBAC-SARS-CoV-REP comprises sequences of a replication competent, non-infectious SARS genome encoding the SARS-CoV replicase and the SARS-CoV N protein. This vector provides a stable, safe and easy to handle basis for producing the virus particles and the vaccines of the present invention. The virus particles and the vaccines may be produced by cloning the at least one further SARS gene into the replicon.

Genomic viral RNA may be used as a starting material to obtain the sequences encoding the further SARS proteins. A cDNA library is prepared from the virus RNAs according to methods known in the art. The full length genes can be reconstructed from the cDNA library. These genes may then be combined with transcriptional regulatory sequences (TRS) and/or translation regulatory sequences (such as internal ribosome entry sites, IRES) and can be inserted into a cloning site of the replicon.

A further embodiment of the invention is directed to the preparation of vaccines with further increased biosafety. According to this aspect, the probability that the nucleic acids of the vaccines of the present invention recombine with a wild-type virus to generate an infectious virus is reduced. For this purpose the present invention is directed to vaccines, wherein the nucleic acid encoding a SARS-CoV replicase has a structure which differs from the genomic structure of the SARS-CoV replicase.

More specifically, a partial sequence of the nucleic acid, which partial sequence encodes a polypeptide essential for replication, is deleted from its position in the native virus and reinserted at a different position of the nucleic acid. The partial sequence may encode at least one of the genes exoribonuclease and/or endoribonuclease and/or 2′-O-ribose methyltransferase (Snijder et al., 2003). These genes are essential for replication of the SARS-CoV nucleic acid. The genes encoding these enzymes are located at the 3′ end of ORF 1b in the structure of the wild-type genome of SARS-CoV. Using the teaching of the present invention one may delete one of the sequences encoding a polypeptide essential for replication from the position, where the gene is found in the genomic structure of the wild-type virus and re-insert the gene at a different position. For example it is possible to re-insert the exoribonuclease gene in the proximity of the 3′end of the construct (such as in the range of 400 to 2000 nucleotides from the 3′ end of the construct), for example next to gene N in the nucleic acids of the present invention. If this nucleic acid recombines with a circulating coronavirus, the risk that the nucleic acid will reincorporate genes encoding the structural proteins and at the same time maintain all essential genes encoding the replicase is significantly reduced.

The nucleic acid may then be used to transfect a helper cell line expressing the essential SARS proteins not encoded by the nucleic acid. Alternatively, a host cell may be transfected with the nucleic acid as described above and a helper virus or other nucleic acid expressing the essential SARS proteins. In this manner the nucleic acid encoding SARS-CoV replicase, SARS-CoV N protein and at least one further SARS-CoV protein will associate with the further essential SARS proteins to form the virus particle of the vaccines of the present invention.

The vaccines can be obtained by producing a virus particle as outlined above and formulating the same into a vaccine, for example by mixing the same with a carrier, adjuvants and/or excipient.

The testing of the vaccines preferably comprises a sequence of several steps. For example the immunogenicity of SARS-CoV VLPs may be tested by producing them in tissue culture of Vero cells. The antigens obtained can be partially purified by conventional procedures including centrifugation and it can be tested whether these induce SARS-CoV neutralizing antibodies in mice and rabbits.

Selected vaccines may further be tested for protection in animal systems, such as BalB/c mice, ferrets and macaques (using conventional methods as described for example in Enserink, 2003; Yang et al., 2004; ter Meulen et al., 2004; Martina et al., 2003; and Kuiken et al., 2004).

The animal model systems can be sequentially used for vaccine efficacy and preliminary safety testing. First, vaccine candidates can be tested in the mice model, then in the ferret SARS-CoV model (Martina et al., 2003). Further decisions can be based on protection against SARS-CoV challenge, the selected vaccine candidates may then be tested in macaques. To achieve this goal, a virus stock can be titrated in young adult cynomolgus macaques. In first experiments increasing logarithmic dilutions of this virus stock (10⁰, 10¹, 10², 10³, 10⁴, 10⁵ and 10⁶ TCID₅₀) should be used to infect macaques intra-tracheally (n=2 per dilution). The clinical, virological, gross pathological and immunological (neutralizing antibodies, cytokine profiles in plasma and cells by mRNA analyses) data may be collected systematically from these SARS-CoV-infected animals, according to protocols that have been established previously (Kuiken et al., 2004).

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows the genetic structure of SARS-CoV Urbani strain. Letters and number inside the boxes indicate the viral genes. L, leader sequence; UTR, untranslated region. Relevant restriction sites are indicated.

FIG. 2 shows the construction of the intermediate plasmid pBAC-SARS-CoV 5′ 3′. A PCR-fragment containing the 5′ end 678 nt and the 3′ end 973 nt of the genome of SARS-CoV Urbani strain, flanked at its left end by the cytomegalovirus (CMV) immediate-early promoter and at its right end by a poly (A) tail followed by the hepatitis delta virus ribozyme (Rz) and the bovine growth hormone (BGH) termination and polyadenylation sequences, is cloned in BAC. To facilitate the assembly of SARS-CoV replicon, a multicloning site containing the restriction sites Cla I, Mlu I, Pme I, and Bam HI, is cloned downstream of the 5′ end viral sequence.

FIG. 3 shows the generation of the SARS-CoV derived replicon pBAC-SARS-CoV-REP. The strategy for the construction of a SARS-CoV replicon is illustrated. SARS-CoV replicon is assembled by sequential cloning of subgenomic overlapping cDNA fragments generated by RT-PCR, into plasmid pBAC-SARS-CoV 5′ 3′. The genetic map of SARS-CoV replicon is shown at the bottom. Relevant restriction sites used in the cloning step are indicated. Rep 1a, Rep 1b, and N indicate the viral genes. CMV, CMV immediate-early promoter; TRS N, natural TRS of N gene; pA, tail of 25 A residues; Rz, hepatitis delta virus ribozyme; BGH, bovine growth hormone termination and polyadenylation sequences.

FIG. 4 shows the functional analysis of SARS-CoV derived replicon. The genetic structure of the SARS-CoV replicon is illustrated at the top. The N gene TRS, the core sequence (italic letters), and the relevant restriction sites are indicated. L, leader sequence; UTR, untranslated region. BHK-21 and human 293T cells are mock transfected (MOCK) or transfected with SARS-CoV replicon (SARS-CoV-REP) or a non-replicative cDNA clone (GFP-NR) using Lipofectamine 2000. Total RNA is isolated at 24 hpt and analyzed by RT-PCR with specific oligonucleotides to detect gene N mRNA. Duplicate RT-PCR products amplified in parallel were resolved by electrophoresis in 1% agarose gels.

FIG. 5 shows the relocalization of an essential gene (E) to reduce the probability of rescue of a full-length infectious coronavirus from a replicon. In the top bar, a simplified scheme of the replicon is shown, including the position of an essential gene (ε), that could be the exoribonuclease gene of ORF 1b. This gene could be deleted in its original position (Δε, middle bar) and relocalized next to gene N. In the event of a potential recombination between the new replicon and a full-length coronavirus with the genome represented in the bottom bar, the new virus generated will not include the essential gene E, and therefore it will not be infectious.

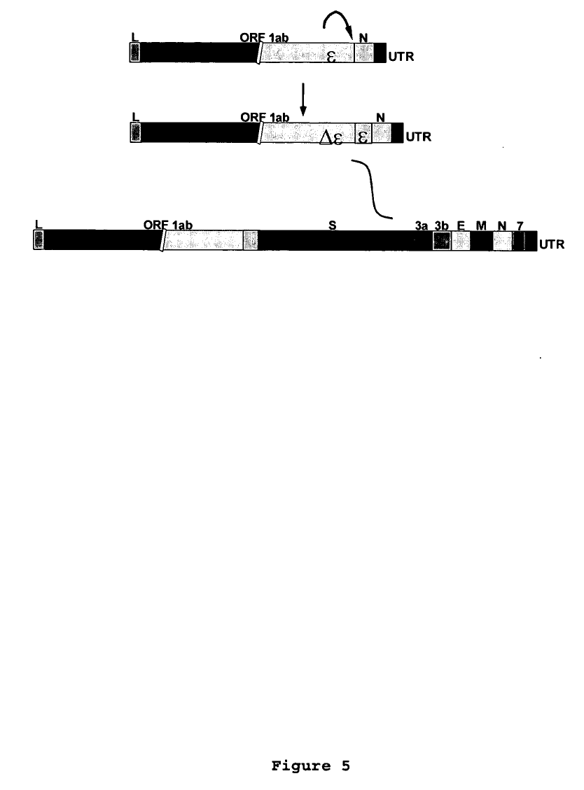

FIG. 6 shows the role of SARS-CoV RNA processing enzymes in RNA synthesis. (A) Schematic representation of the different single deletion mutant replicons. The conserved domains described by Snijder (2003) are indicated by dark bars. In addition, a deletion mutant lacking the three domains (A3) was also generated. (B) Functional analysis of mutant replicons. Expression of gene N mRNA was used to study replicon activity in BHK and 293T cells by RT-PCR analysis. M, Mock.

The following examples illustrate but do not limit the embodiments of the invention.

### Examples

The following cells, viruses, plasmids and bacteria strains were used: Cells and viruses. Baby hamster kidney cells (BHK-21) and human 293T cells were purchased from the American Collection of Cell Cultures (ATCC). All cells were maintained in DMEM supplemented with 10% fetal bovine serum and antibiotics at 37° C. in a CO₂ incubator. The genomic RNA of Urbani strain was obtained from the Center for Disease Control and Prevention (CDC) after signature of a material transfer agreement. Plasmids and bacteria strains. Plasmid pBeloBAC11 (Wang et al., 1997) was kindly provided by H. Shizuya and M. Simon (California Institute of Technology, Pasadena, Calif.). Escherichia coli DH10B strain was obtained from GIBCO/BRL. DH10B cells were transformed by electroporation at 25 μF, 2.5 kV, and 200Ω with a Gene Pulser unit (Bio-Rad) according to the manufacturer's instructions. Plasmid DNA was isolated with the Qiagen (Chatsworth, Calif.) Large Construct Kit according to the manufacturer's specification.

### Example 1

### Construction of a SARS-CoV Replicon cDNA

A cDNA that contained the untranslated 5′ and 3′ end of the Urbani genome and the replicase and N genes, was cloned as a BAC under the control of a CMV promoter.

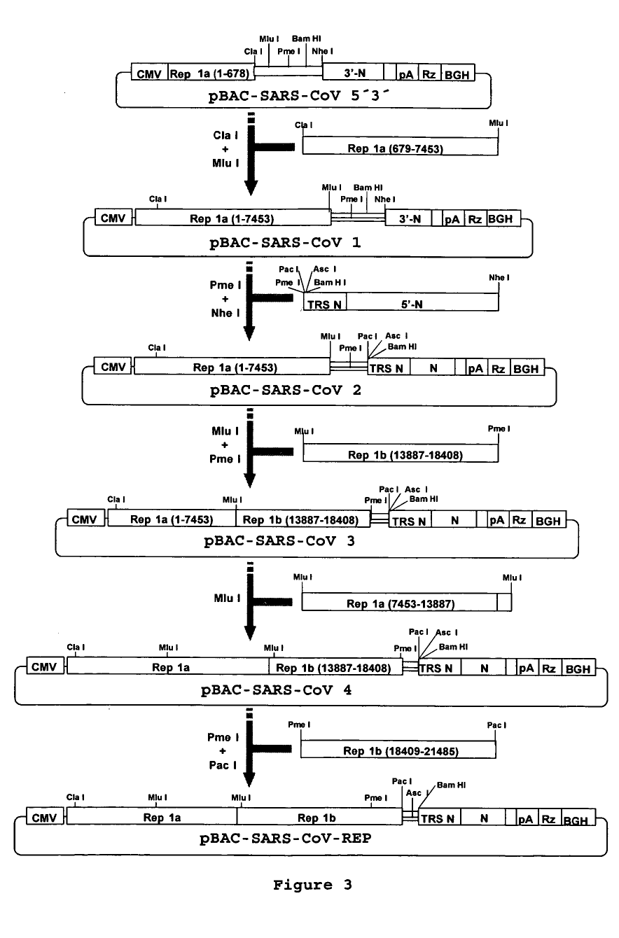

Additionally, a multicloning site containing unique restriction sites Pac I, Asc I, and Bam HI, was cloned downstream of the replicase gene to allow cloning of heterologous genes (FIG. 3). This approach use a two-step amplification system that couples replicon RNA transcription in the nucleus from the CMV promoter with a second amplification step in the cytoplasm driven by the viral polymerase. The plasmid pBAC-SARS-CoV-REP, encoding the SARS-CoV replicon, was stable for at least 180 generations during its propagation in DH10B cells, as determined by restriction endonuclease analysis.

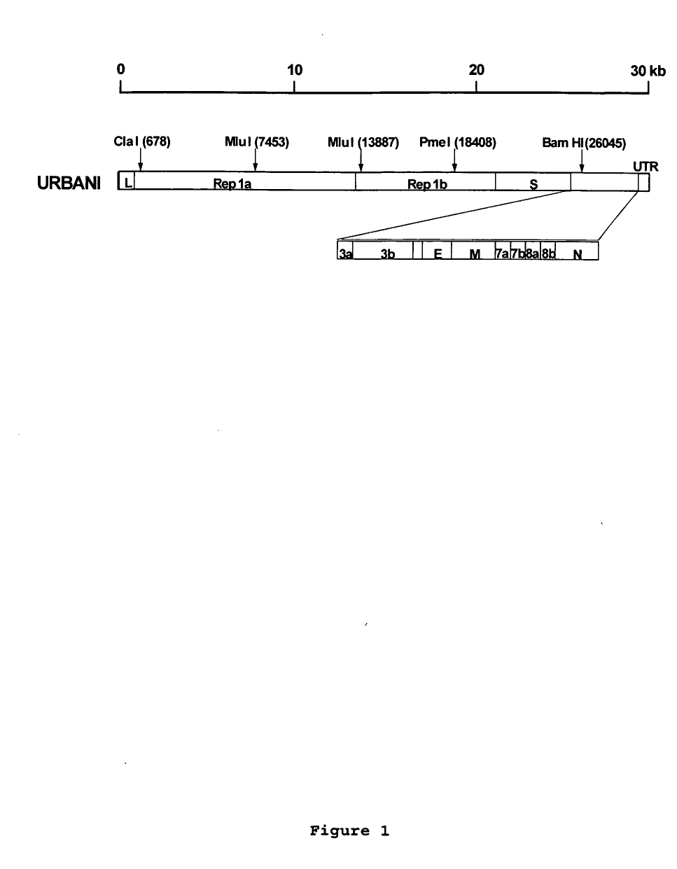

In a first step the appropriate restriction sites in the viral genome that can be used in the engineering of the replicon were identified (FIG. 1).

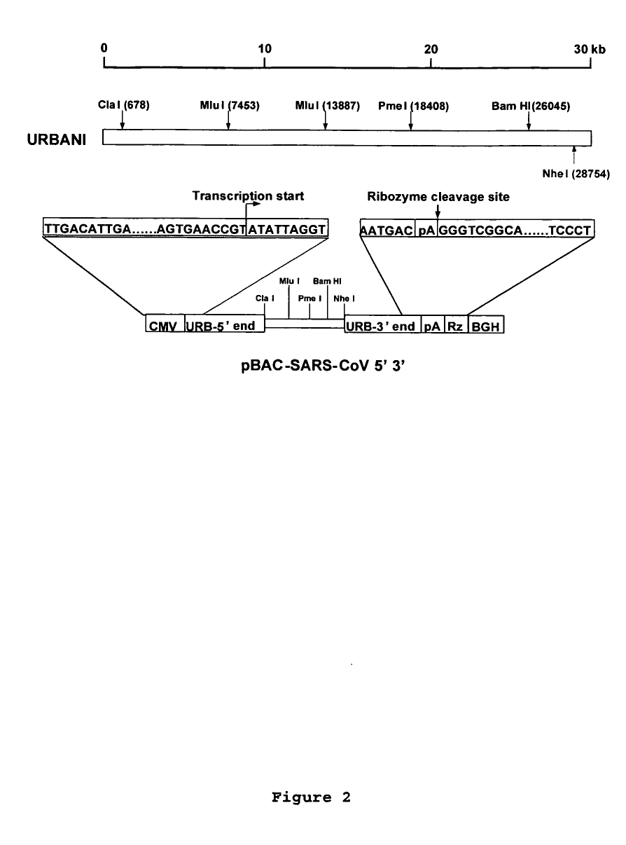

In a second step the intermediate plasmid pBAC-SARS-CoV 5′ 3′ was generated as the basis to construct the SARS-CoV replicon (FIG. 2). This plasmid contained the first 678 nt of the 5′end of the Urbani strain genome under the control of the cytomegalovirus (CMV) immediate-early promoter and the last 973 nt of the genome followed by a 25-bp synthetic poly (A), the hepatitis delta virus ribozyme, and the bovine growth hormone termination and polyadenylation sequences to make an accurate 3′ end. Additionally, a multicloning site containing the restriction sites Cla I, Mlu I, Pme I, and Bam HI, selected in the first step, was cloned in between the SARS-CoV sequences to allow the assembly of the SARS-CoV replicon.

In a third step the overlapping SARS-CoV cDNA subclones were generated by RT-PCR and the replicon was assembled using the restriction sites selected. Using the pBAC-SARS-CoV 5′ 3′ as starting point, a SARS-CoV replicon was engineered following the strategy described in FIG. 3. The plasmid pBAC-SARS-CoV-REP contained the untranslated 5′ and 3′ ends of the Urbani genome, the replicase gene followed by a multicloning site containing unique restriction sites Pac I, Asc I, and Bam HI, to allow cloning and expression of heterologous genes, and the nucleoprotein (N) gene under the control of its natural TRS.

### Example 2

### Analysis of Cloned cDNA Stability

The stability of the viral sequences cloned into pBeloBAC11 was analyzed by studying the restriction endonuclease pattern at different passages. Bacteria transformed with recombinant plasmid were grown in 10 ml of LB containing 12.5 μg/ml chloramphenicol at 37° C. Cells from these primary cultures (considered passage 0) were propagated serially by diluting 10⁶-fold daily. Each passage was considered to represent about 20 generations.

### Example 3

### Sequence Analysis

DNA was sequenced using an automatic 373 DNA sequencer (Applied Biosystem) using fluorochrome labeled dideoxynucleotides and temperature resistant DNA polymerase (Perkin Elmer).

### Example 4

### Transfection of SARS-CoV Replicon cDNA

BHK-21 and 293T cells were grown to 95% confluence on 35-mm-diameter plates and transfected with 5 μg of the SARS-CoV replicon, using 6 μg of Lipofectamine 2000 (Invitrogen) according to the manufacturer's specifications.

### Example 5

### RNA Isolation and RT-PCR Analysis

Total intracellular RNA was extracted at 24 h post-transfection (hpt) with the RNeasy Mini Kit (Qiagen) and used as template for RT-PCR analysis of gene N mRNA transcription. RT reactions were performed with Moloney murine leukaemia virus reverse transcriptase (Ambion) and the antisense primer URB-28630RS (5′-TGCTTCCCTCTGCGTAGAAGCC-3′) (SEQ ID NO:7), complementary to nucleotides 510 to 531 of gene N. The cDNAs generated were amplified by PCR using the reverse primer URB-28630RS and the forward primer URB-29VS (5′-GCCAACCAACCTCGATCTCTTG-3′) (SEQ ID NO:8), spanning nucleotides 29 to 50 of the Urbani leader sequence. For the quantitative analysis by real-time RT-PCR of gene N mRNA, the primers used for RT (URB-28163RS, 5′-TGGGTCCACCAAATGTAATGC-3′ (SEQ ID NO:9), complementary to nucleotides 43 to 63 of gene N) and PCR (reverse primer URB-28163RS and the forward primer URB-27VS, 5′-AAGCCAACCAACCTCGATCTC-3′ (SEQ ID NO:10), spanning nucleotides 27 to 47 of the Urbani leader sequence) were designed using the Primer Express software (Applied Biosystems). The SYBR Green PCR master mix was used in the PCR step following the manufacturer's specifications (Applied Biosystems).

### Example 6

### Analysis of the SARS-CoV Derived Replicon

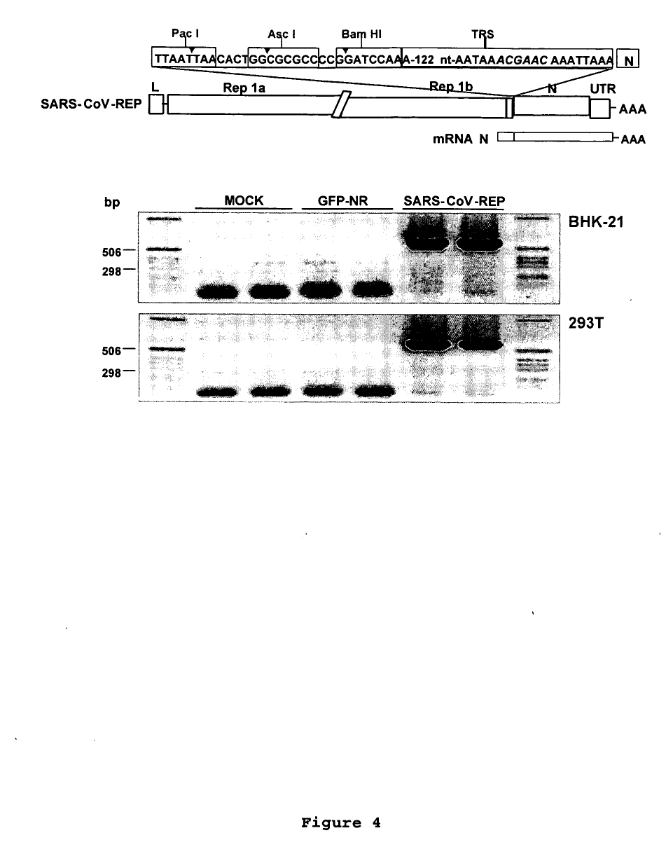

The SARS-CoV derived replicon was functional in several cell lines. Expression of gene N mRNA was used to study replicon activity by RT-PCR analysis. BHK-21 and human 293T cells were transfected with either SARS-CoV replicon or a non-replicative cDNA clone using Lipofectamine 2000. Total intracellular RNA was extracted at 24 hpt and used as template for RT-PCR analysis of gene N mRNA transcription using specific oligonucleotides. High levels of gene N mRNA were detected in both BHK-21 and 293T cells transfected with the SARS-CoV replicon (FIG. 4), showing that the replicon is active in these cell lines. A quantitative analysis of gene N mRNA in transfected 293T and BHK-21 cells was performed by real-time RT-PCR using the primers described in Example 5. The SARS-CoV replicon activity was 8-fold higher in 293T cells than in BHK-21 cells. This activity increase could be explained by a higher replication level of SARS-CoV replicon in human 293T cells or simply because the transfection efficiency of 293T cells is twice higher than that of BHK-21 cells.

The role of the N protein in SARS-CoV replicon activity was analyzed. To achieve this objective, a SARS-CoV replicon lacking the N gene was constructed and the replicon activity compared with that of the replicon expressing the N gene. A basal activity of the replicon lacking N gene was detected, but replicon activity increased more than 100-fold when N protein was present. These data indicated that N protein plays an important role as an enhancer of coronavirus replicon activity.

Bacteria containing the plasmid pBAC-SARS-CoV-REP comprising sequences of a replication competent, non-infectious SARS genome encoding the SARS-CoV replicase and the SARS-CoV N protein were deposited on Sep. 1, 2004 with the Colección Española de Cultivos (Tipo, CECT in Valencia, Spain). The deposit was given the provisional accession number 7020.

### Example 7

### Role of SARS-CoV RNA Processing Enzymes in RNA Synthesis

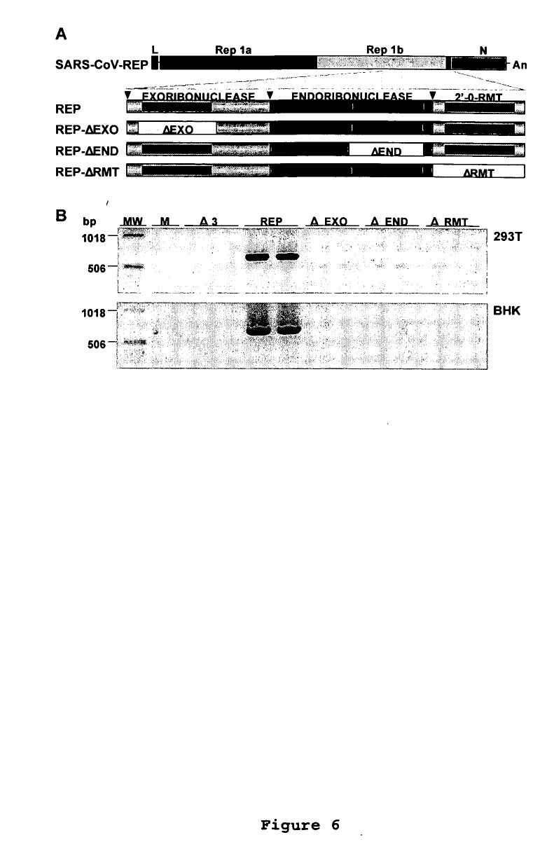

Using the pBAC-SARS-CoV-REP replicon the role in coronavirus RNA synthesis of the recently described RNA processing enzymes exoribonuclease, endoribonuclease, and 2′-O-ribose methyltransferase (Snijder et al., 2003) was investigated. To that, single deletion mutant replicons, in which the conserved domain for each enzyme was deleted, and a mutant lacking the three domains were generated (FIG. 6A). Replicon activity of these constructs was analyzed in both BHK and 293T cells and only the wild type replicon was functional (FIG. 6B), indicating that these RNA processing enzymes are essential for SARS-CoV RNA synthesis.

### BIBLIOGRAPHY

Almazán, F., J. M. Gonzalez, Z. Pénzes, A. Izeta, E. Calvo, J. Plana-Durán, and L. Enjuanes (2000). Engineering the largest RNA virus genome as an infectious bacterial artificial chromosome. Proc. Natl. Acad. Sci. USA 97:5516-5521.
Alonso, S., Izeta A., Sola I., and Enjuanes L. (2002a). Transcription regulatory sequences and mRNA expression levels in the coronavirus transmissible gastroenteritis virus. J. Virol. 76:1293-1308
Alonso, S., Sola, I., Teifke, J., Reimann, I., Izeta, A., Balach, M., Plana-Durán, J., Moormann, R. J. M., and Enjuanes, L. (2002b). In vitro and in vivo expression of foreign genes by transmissible gastroenteritis coronavirus-derived minigenomes. J. Gen. Virol. 83, 567-579.
Casais, R., V. Thiel, S. G. Siddell, D. Cavanagh, and P. Britton (2001). Reverse genetics system for the avian coronavirus infectious bronchitis virus. J. Virol. 75:12359-12369.
Chang, K.-Y., and Tinoco, I. (1994). Characterization of a “kissing” hairpin complex derived from the human immunodeficiency virus genome. Proc. Natl. Acad. Sci. USA 91, 8705-8709.
Drosten, C., S. Gunther, W. Preiser, S. van der Werf, H. R. Brodt, S. Becker, H. Rabenau, M. Panning, L. Kolesnikova, R. A. Fouchier, A. Berger, A. M. Burguiere, J. Cinatl, M. Eickmann, N. Escriou, K. Grywna, S. Kramme, J. C. Manuguerra, S. Muller, V. Rickerts, M. Sturmer, S. Vieth, H. D. Kienk, A. D. Osterhaus, H. Schmitz, and H. W. Doerr (2003). Identification of a novel coronavirus in patients with severe acute respiratory syndrome. N. Engl. J. Med. 348:1967-1976.
Enserink M. (2003). Infectious diseases. SARS researchers report new animal models. Science 302(5643):213.
González, J. M., Z. Pénzes, F. Almazán, E. Calvo, and L. Enjuanes (2002). Stabilization of a full-length infectious cDNA clone of transmissible gastroenteritis coronavirus by insertion of an intron. J. Virol. 76:4655-4661.
Holmes, K. V., and L. Enjuanes (2003). Virology. The SARS coronavirus: a postgenomic era. Science 300:1377-1378.
Izeta, A., Smerdou, C., Alonso, S., Penzes, Z., Méndez, A., Plana-Durán, J., and Enjuanes, L. (1999). Replication and packaging of transmissible gastroenteritis coronavirus-derived synthetic minigenomes. J. Virol. 73, 1535-1545.
Kiyono et al. (1996). Mucosal Vaccines. Academic Press, New York.
Kuiken T, van den Hoogen B G, van Riel D A, Laman J D, van Amerongen G, Sprong L, Fouchier R A, Osterhaus A D. (2004). Experimental human metapneumovirus infection of cynomolgus macaques (Macaca fascicularis) results in virus replication in ciliated epithelial cells and pneumocytes with associated lesions throughout the respiratory tract. Am J Pathol. 164:1893.
Marra, M. A., S. J. Jones, C. R. Astell, R. A. Holt, A. Brooks-Wilson, Y. S. Butterfield, J. Khattra, J. K. Asano, S. A. Barber, S. Y. Chan, A. Cloutier, S. M. Coughlin, D. Freeman, N. Girn, O. L. Griffith, S. R. Leach, M. Mayo, H. McDonald, S. B. Montgomery, P. K. Pandoh, A. S. Petrescu, A. G. Robertson, J. E. Schein, A. Siddiqui, D. E. Smailus, J. M. Stott, G. S. Yang, F. Plummer, A. Andonov, H. Artsob, N. Bastien, K. Bernard, T. F. Booth, D. Bowness, M. Czub, M. Drebot, L. Fernando, R. Flick, M. Garbutt, M. Gray, A. Grolla, S. Jones, H. Feldmann, A. Meyers, A. Kabani, Y. Li, S. Normand, U. Stroher, G. A. Tipples, S. Tyler, R. Vogrig, D. Ward, B. Watson, R. C. Brunham, M. Krajden, M. Petric, D. M. Skowronski, C. Upton, and R. L. Roper (2003). The genome sequence of the SARS-associated coronavirus. Science 300:1399-1404.
Martina B E, Haagmans B L, Kuiken T, Fouchier R A, Rimmelzwaan G F, Van Amerongen G, Peiris J S, Lim W, Osterhaus A D. (2003). Virology: SARS virus infection of cats and ferrets. Nature 425:915.
Rota, P. A., M. S. Oberste, S. S. Monroe, W. A. Nix, R. Campagnoli, J. P. Icenogle, S. Penaranda, B. Bankamp, K. Maher, M. H. Chen, S. Tong, A. Tamin, L. Lowe, M. Frace, J. L. DeRisi, Q. Chen, D. Wang, D. D. Erdman, T. C. Peret, C. Burns, T. G. Ksiazek, P. E. Rollin, A. Sanchez, S. Liffick, B. Holloway, J. Limor, K. McCaustland, M. Olsen-Rasmussen, R. Fouchier, S. Gunther, A. D. Osterhaus, C. Drosten, M. A. Pallansch, L. J. Anderson, and W. J. Bellini (2003). Characterization of a novel coronavirus associated with severe acute respiratory syndrome. Science 300:1394-1399.
Sawicki, S. G., and D. L. Sawicki (1998). A new model for coronavirus transcription. Adv. Exp. Med. Biol. 440:215-219.
Sawicki & Sawicki (1990). Coronavirus transcription: subgenomic mouse hepatitis virus replicative intermediates function in RNA synthesis. J. Virol. 64(3):1050-6.
Sethna, P. B., Hung, S.-L., and Brian, D. A. (1989). Coronavirus subgenomic minus-strand RNAs and the potential for mRNA replicons. Proc. Natl. Acad. Sci. USA 86, 5626-5630.
Siddell, S. G. (1995). “The Coronaviridae.” The Viruses (H. Fraenkel-Conrat, and R. R. Wagner, Eds.) Plenum Press, New York.
Snijder, E. J., Bredenbeek, P. J., Dobbe, J. C., Thiel, V., Ziebuhr, J., Poon, L. L., Guan, Y., Rozanov, M., Spaan, W. J., and Gorbalenya, A. E., 2003. Unique and conserved features of genome and proteome of SARS-coronavirus, an early split-off from the coronavirus group 2 linage. J. Mol. Biol. 331: 991-1004.
Sanchez, C. M., Izeta, A., Sánchez-Morgado, J. M., Alonso, S., Sola, I., Balasch, M., Plana-Durán, J. and Enjuanes, L. (1999). Targeted recombination demonstrates that the spike gene of transmissible gastroenteritis coronavirus is a determinant of its enteric tropism and virulence. J. Virol. 73:7607-7618.
ter Meulen J, Bakker A B, van den Brink E N, Weverling G J, Martina B E, Haagmans B L, Kuiken T, de Kruif J, Preiser W, Spaan W, Gelderblom H R, Goudsmit J, Osterhaus A D. (2004). Human monoclonal antibody as prophylaxis for SARS coronavirus infection in ferrets. Lancet. 26;363(9427):2139-41.
Thiel, V., J. Herold, B. Schelle, and S. G. Siddell (2001). Infectious RNA transcribed in vitro from a cDNA copy of the human coronavirus genome cloned in vaccinia virus. J. Gen. Virol. 82:1273-1281.
Thiel, V., K. A. Ivanov, A. Putics, T. Hertzig, B. Schelle, S. Bayer, B. Weibrich, E. J. Snijder, H. Rabenau, H. W. Doerr, A. E. Gorbalenya and J. Ziebuhr (2003). Mechanism and enzymes involved in SARS coronavirus genome expression. J. Gen. Virol. 84: 2305-2315.
Toka, F. N. et al. (2004). Molecular adjuvants for mucosal immunity. Immunol Rev. 199:100-112.
van der Most, R. G., and Spaan, W. J. M. (1995). Coronavirus replication, transcription, and RNA recombination. In “The Coronaviridae” (S. G. Siddell, Ed.), pp. 11-31. Plenum Press, New York.
Wang, K., C. Boysen, H. Shizuya, M. I. Simon and L. Hoods (1997). Complete nucleotide sequence of two generations of a bacterial artificial chromosome cloning vector. BioTechniques 23: 992-994.
Yang Z Y, Kong W P, Huang Y, Roberts A, Murphy B R, Subbarao K, Nabel G J. (2004). A DNA vaccine induces SARS coronavirus neutralization and protective immunity in mice. Am J Pathol. 164:1893-900.
Yount, B., K. M. Curtis, and R. S. Baric (2000). Strategy for systematic assembly of large RNA and DNA genomes: transmissible gastroenteritis virus model. J. Virol. 74:10600-10611.
Yount, B., M. R. Denison, S. R. Weiss, and R. S. Baric (2002). Systematic assembly of a full-length infectious cDNA of mouse hepatitis virus strain A59. J. Virol. 76:11065-11078.
Yount, B., K. M. Curtis, E. A. Fritz, L. E. Hensley, P. B. Jahrling, E. Prentice, M. R. Denison, T. W. Geisbert, and R. S. Baric (2003). Reverse genetics with a full-length infectious cDNA of severe acute respiratory syndrome coronavirus. Proc. Natl. Acad. Sci. USA 100:12995-13000.
Ziebuhr, J., E. J. Snijder, and A. E. Gorbalenya (2000). Virus-encoded proteinases and proteolytic processing in the Nidovirales. J. Gen. Virol. 81:853-879.
Zuniga, S., I. Sola, S. Alonso, and L. Enjuanes (2004). Sequence motifs involved in the regulation of discontinuous coronavirus subgenomic RNA synthesis. J. Virol. 78:980-994.

1. Vaccine comprising a virus particle comprising a nucleic acid and one or several coat proteins, wherein
(a) the nucleic acid encodes SARS-CoV replicase, SARS-CoV N protein and at least one further SARS-CoV protein; and
(b) the one or several coat proteins are SARS-CoV coat proteins or proteins with a homology of at least 60% to SARS-CoV coat proteins necessary to allow the virus particle to infect human or animal cells,
wherein the nucleic acid is replication competent but not infectious.

2. Vaccine according to claim 1, wherein the SARS-CoV replicase is encoded by SEQ ID NO: 1 (Rep1a) and SEQ ID NO:2 (Rep1b) or a nucleic acid having a homology of at least 60% to the SEQ ID NOs: 1 or 2.

3. Vaccine according to claim 1, wherein the SARS-CoV N protein is encoded by SEQ ID NO: 3 or a nucleic acid having a homology of at least 60% to the SEQ ID NO: 3.

4. Vaccine according to claim 1, wherein the nucleic acid further encodes SARS-CoV spike protein.

5. Vaccine according to claim 4, wherein the SARS-CoV spike protein is encoded by SEQ ID NO: 4 or a nucleic acid having a homology of at least 60% to the SEQ ID NO: 4.

6. Vaccine according to claim 5, wherein the nucleic acid does not encode any other SARS-CoV protein except for SARS-CoV replicase, SARS-CoV N protein and SARS-CoV spike protein.

7. Vaccine according to claim 1, wherein the nucleic acid further encodes SARS-CoV M protein.

8. Vaccine according to claim 7, wherein the SARS-CoV M protein is encoded by SEQ ID NO: 5 or a nucleic acid having a homology of at least 60% to the SEQ ID NO: 5.

9. Vaccine according to claim 8, wherein the nucleic acid does not encode any other SARS-CoV protein except for SARS-CoV replicase, SARS-CoV N protein and SARS-CoV M protein.

10. Vaccine according to claim 1, wherein the nucleic acid further encodes SARS-CoV E protein.

11. Vaccine according to claim 10, wherein the SARS-CoV E protein is encoded by SEQ ID NO: 6 or a nucleic acid having a homology of at least 60% to the SEQ ID NO: 6.

12. Vaccine according to claim 11, wherein the nucleic acid does not encode any other SARS-CoV protein except for SARS-CoV replicase, SARS-CoV N protein and SARS-CoV E protein.

13. Vaccine according to claim 1, wherein the nucleic acid further encodes SARS-CoV M protein, SARS-CoV E protein and SARS-CoV spike protein.

14. Vaccine according to claim 13, wherein the nucleic acid does not encode any other SARS-CoV protein except for SARS-CoV replicase, SARS-CoV N protein, SARS-CoV M protein, SARS-CoV E protein and SARS-CoV spike protein.

15. Vaccine according to claim 1, wherein the nucleic acid encodes SARS-CoV proteins capable of associating into SARS-CoV virus like particles.

16. Vaccine according to claim 15, wherein the nucleic acid encodes SARS-CoV protein M, SARS-CoV spike protein and SARS-CoV protein E or proteins with a homology of at least 60% to SARS-CoV M and SARS-CoV E protein.

17. Vaccine according to claim 1, wherein administration of the vaccine to a mammal gives rise to an immune response that reduces or eliminates the disease symptoms caused by infection with the SARS virus.

18. Vaccine according to claim 1, wherein the vaccine is administered to a human or an animal.

19. Vaccine according to claim 18 wherein the vaccine is administered intramuscularly, intranvenously, subcutaneously or intranasally.

20. Vaccine according to claim 1, wherein the vaccine further comprises pharmaceutically acceptable carrier and/or adjuvants.

21. Vaccine according to claim 20, wherein the adjuvants is selected from the list consisting of cytokines, chemokines granulocyte/macrophage colony-stimulating factor, interleukin-2 (IL-2), IL-12, IL-18, IL-15, IL-23 and CpG.

22. Vaccine according to claim 1, wherein the nucleic acid encoding a SARS-CoV replicase has a structure which differs from the genomic structure of the SARS-CoV replicase.

23. Vaccine according to claim 1, wherein a partial sequence of the nucleic acid, which partial sequence encodes a polypeptide essential for replication, is deleted from its natural position and reinserted at a different position of the nucleic acid.

24. Vaccine according to claim 23, wherein the partial sequence encodes the exoribonuclease and/or endoribonuclease and/or 2′-O-ribose methyltransferase.

25. Vaccine according to claim 23, wherein the partial sequence is reinserted in at about 400 to about 2000 nucleotides from the 3′ end of the viral nucleic acids.


SEQUENCE LISTING (10 sequences)

SEQ ID NO: 1 - DNA, 13149 bp, SARS-CoV
atggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagttcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgtttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaa

SEQ ID NO: 2 - DNA, 8088 bp, SARS-CoV
cgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgctactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggagctcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaa

SEQ ID NO: 3 - DNA, 1269 bp, SARS-CoV
atgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataa

SEQ ID NO: 4 - DNA, 3768 bp, SARS-CoV
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 5 - DNA, 666 bp, SARS-CoV
atggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacaccccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaa

SEQ ID NO: 6 - DNA, 231 bp, SARS-CoV
atgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaa

SEQ ID NO: 7 - DNA, 22 bp, Artificial Sequence
  oligonucleotide primer
tgcttccctctgcgtagaagcc

SEQ ID NO: 8 - DNA, 22 bp, Artificial Sequence
  oligonucleotide primer
gccaaccaacctcgatctcttg

SEQ ID NO: 9 - DNA, 21 bp, Artificial Sequence
  oligonucleotide primer
tgggtccaccaaatgtaatgc

SEQ ID NO: 10 - DNA, 21 bp, Artificial Sequence
  oligonucleotide primer
aagccaaccaacctcgatctc